(19) United States
(12) Patent Application Publication
(10) Pub. No.: US 2015/0352361 A1
(43) Pub. Date: Dec. 10, 2015

(54) ARTIFICIAL RETINAL SYSTEM

(71) Applicant: National Chiao Tung University, Hsinchu City (TW)

(72) Inventors: Chung-Yu Wu, Hsinchu City (TW); Po-Kang Lin, Taipei City (TW); Chuan-Chin Chiao, Taipei City (TW); Jui-Wen Pan, Hsinchu City (TW)

(73) Assignee: National Chiao Tung University

(21) Appl. No.: 14/730,360

(22) Filed: Jun. 4, 2015

(30) Foreign Application Priority Data
Jun. 9, 2014 (TW) 103119924

(51) Int. Cl. A61N 1/36

(52) U.S. Cl. CPC A61N 1/36046

(57) ABSTRACT

An artificial retinal system includes an optical device and a retinal implant device. The optical device includes an image generator for generating (M) projected images, each having (N) projected blocks with (M) identical projected sub-blocks, based differently on an external image, and output ting the projected images in sequence through optical projection. The projected sub-blocks of each projected block of each projected linage are associated with a corresponding original sub-block of a corresponding original block. The retinal implant device includes a pixel array with (N) pixel units, each generating (M) electrical stimulus signals based on a respective one of the projected blocks, and combines the electrical stimulus signals to obtain a total electrical stimulus signal.

### CROSS-REFERENCE TO RELATED APPLICATION

This application claims priority of Taiwanese Application No. 103119924, filed on Jun. 9, 2014, the contents of which are hereby incorporated by reference.

### FIELD OF THE INVENTION

This invention relates to an optical device, and sore particularly to an artificial retinal system.

### BACKGROUND OF THE INVENTION

A conventional artificial retina includes a solar cell that receives and converts an optical image into electrical signal for stimulating the retina, such that visions of patients blinded as a result of degeneration of photoreceptors can be restored. In the prior art, the relatively small amount of stimuli due to insufficient power supply of the solar cell is an issue.

### SUMMARY OF THE INVENTION

Therefore, an object of the present invention is to provide an artificial retinal system that can overcome the aforesaid drawbacks associated with the prior art.

According to this invention, the artificial retinal system may include an optical device and a retinal implant device.

The optical device includes an image generator. The image generator receives an external image, generates a number (M) of projected images based differently on the external image, and outputs the projected images in sequence through optical projection. The external image has a number (N) of original blocks, each of which has a number (M) of original sub-blocks. Each of the projected images has a number (N) of projected blocks, each of which has a number (M) of identical projected sub-blocks. The projected sob-blocks of each of the projected blocks of each of the projected images are associated with a corresponding one of the original sub-blocks of a corresponding one of the original blocks. N and M are positive integers greater than 1.

The retinal implant device includes a pixel array. The pixel array includes a number (N) of pixel units respectively receiving the projected blocks of one of the projected images. Each of the pixel units generates, based on the respective one of the projected blocks thus received, a number (M) of electrical stimulus signals that are respectively associated with the projected sub-blocks of the respective one of the projected blocks, and combines the electrical stimulus signals to obtain a total electrical stimulus signal.

### BRIEF DESCRIPTION OF THE DRAWINGS

Other features and advantages of the present invention will become apparent in the following detailed description of the embodiment of this invention, with reference to the accompanying drawings, in which:

FIG. 1 is a schematic diagram illustrating the embodiment of an artificial retinal system according to this invention;

FIG. 2 is a block diagram illustrating the embodiment;

FIG. 3A is a schematic diagram illustrating an external image received by the embodiment;

FIG. 3B is a schematic diagram illustrating a projected image of the embodiment;

FIG. 4 is a circuit block diagram illustrating a pixel unit of the embodiment; and

FIG. 5 is a schematic diagram illustrating an linage processing flow of the embodiment.

### DETAILED DESCRIPTION OF THE EMBODIMENT

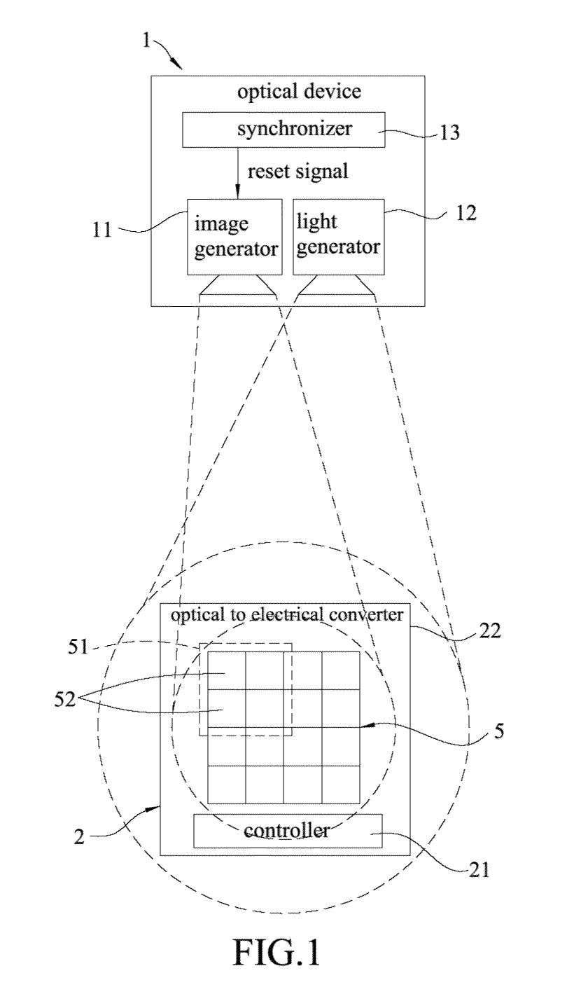

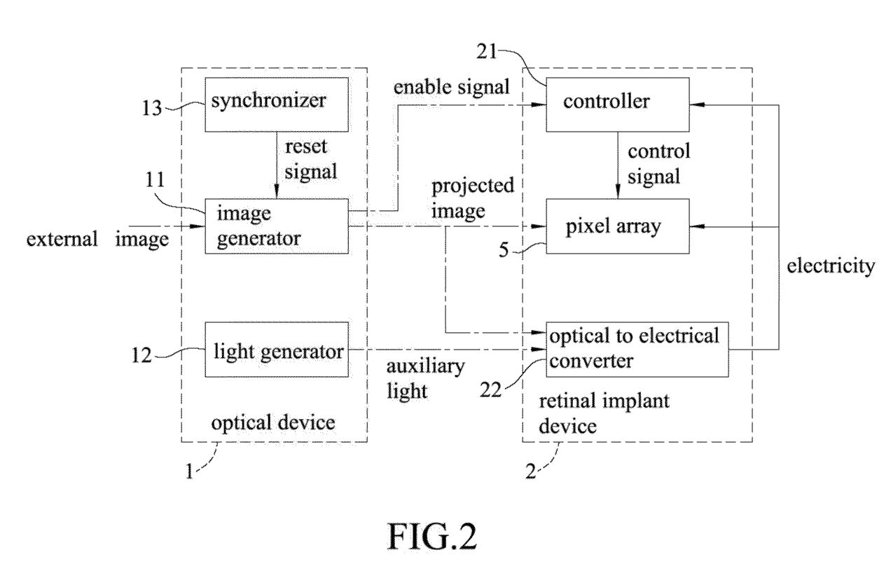

Referring to FIGS. 1 and 2, the embodiment of an artificial retinal system according to this invention includes an optical device 1 and a retinal implant device 2.

The optical device 1 includes a light generator 12, a synchronizer 13 and an linage generator 11.

The light generator 12 emits an auxiliary light.

The synchronizer 13 detects an occurrence of a predetermined event, and outputs a reset signal when the occurrence of the predetermined event is detected. In this embodiment, the predetermined event is a transition from opening of an eye into dosing of the eye.

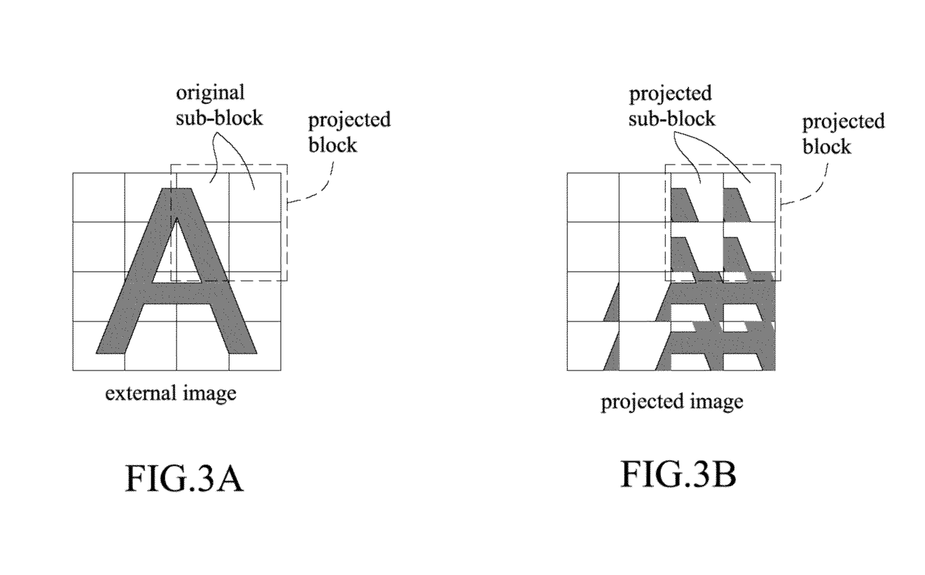

The image generator 11 receives an external image, generates a number (M) of projected images based differently on the external image, and outputs the projected images in sequence through optical projection. For instance, the projected images may be output ted at periodic intervals, As shown in FIG. 3A, the external linage has a number (N) of original blocks, each of which has a number (M) of original sub-blocks. As shown in FIG. 3B, each of the projected images has a number (N) of projected blocks, each of which has a number (M) of identical projected sub-blocks. The projected sub-blocks of each of the projected blocks of each of the projected images are associated with a corresponding one of the original sub-blocks of a corresponding one of the original blocks. N and M are positive integers greater than 1. Specifically, the projected sub-blocks of an i^{th} one of the projected blocks of a j one of the projected images generated by the image generator 11 are associated with a j^{th} one of the original sub-blocks of an i^{th} one of the original blocks of the external image, where i=(1, 2, . . . , N) and j=(1, 2, . . . , M).

The image generator 11 is coupled to the synchronizer 13 for receiving the reset signal therefrom, and re-receives the external, image upon receipt of the reset signal.

The retinal implant device 2 in this embodiment is an implantable biomedical chip to be disposed in a sub-retinal of a patient's eyeball through surgical operation. The retinal implant device 2 includes a controller 21, an optical to electrical converter 22 and a pixel array 5.

The optical to electrical converter 22 is coupled to the controller 21 and the pixel array 5, receives the projected images from the image generator 11 and the auxiliary light from the light generator 12, and converts the projected images and the auxiliary light into electricity for powering the controller 21 and the pixel array 5.

The pixel array 5 includes a number (N) of pixel units 51 respectively receiving the projected blocks of one of the projected images. Each pixel unit 51 generates, based on the received projected block, a number (M) of electrical stimulus signals respectively associated with the projected sub-blocks of the received projected block, and combines the (M) number of the electrical stimulus signals to obtain a total electrical stimulus signal.

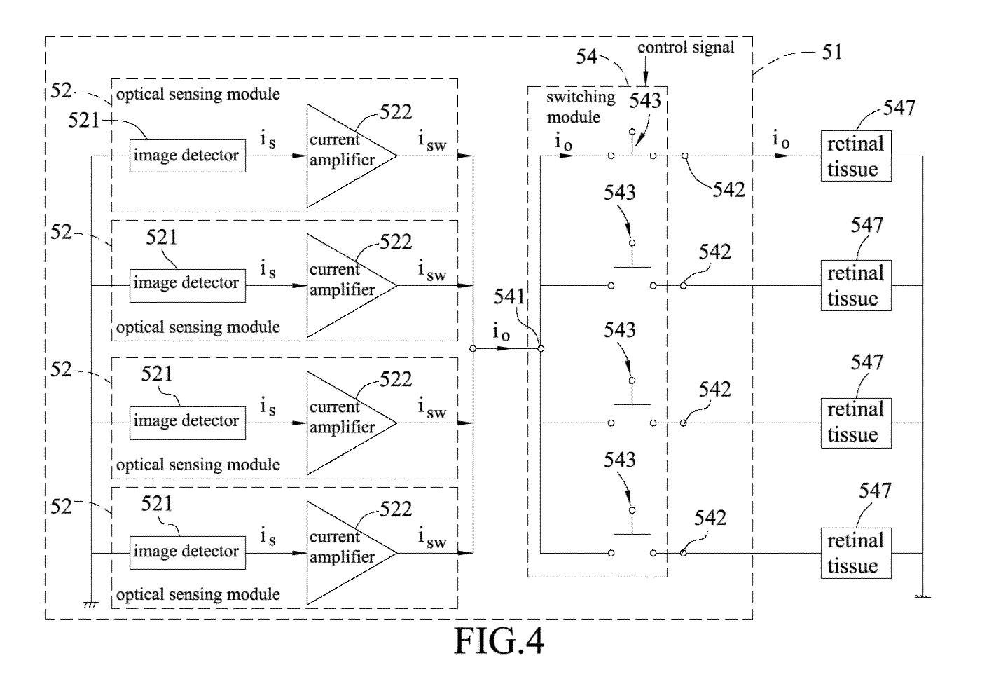

Referring to FIG. 4, in this embodiment, each of the pixel units 51 includes a number (M) of optical sensing modules 52 and a switching module 54. In other words, a total number of the optical sensing modules 52 in the pixel unit 5 is M*N.

Each of the optical sensing modules 52 of each pixel unit 51 receives a respective one of the projected sub-blocks of the projected block received by the corresponding pixel unit 51 (i.e., the received projected block previously mentioned), and generates, based on the received projected sub-block, a respective one of the electrical stimulus signals (i_{sw}). Each of the optical sensing modules 52 includes an image detector 521 and a current amplifier 522.

The image detector 521 receives the respective one of the projected sub-blocks of the received projected block, and converts the received projected sub-block into a current signal (iₛ).

The current amplifier 522 is coupled to the image detector 521 for receiving the current signal (iₛ) therefrom, and amplifies the current signal (iₛ) to obtain the respective one of the electrical stimulus signals (i_{sw}).

The switching module 54 of each pixel unit 51 has an input terminal 541 that is coupled to the optical sensing modules 52 for receiving the electrical stimulus signals respectively therefrom, and a number (M) of output terminals 542 that are respectively disposed in contact with a number (M) of sub-regions 547 of a corresponding one of a number (N) of regions of the retina (also referred to as retinal sub-regions 547). The retinal sub-regions 547 correspond in position respectively to the optical sensing modules 52 of the corresponding pixel unit 51. The electrical stimulus signals (i_{sw}) are combined at the input terminal 541 to obtain the total electrical stimulus signal (iₒ). The switching module 54 is controlled by the controller 21 (see FIG. 2) to selectively output the total electrical stimulus signal (iₒ) at one of the output terminals 542 to stimulate the respective retinal sub-region 547.

Specifically, the controller 21 is enabled by the linage generator 11 to control the switching module 54 of each of the pixel units 5 to output the total electrical stimulus signal (iₒ) at a selected one of the output terminals 542. The selected one of the output terminals 542 corresponds to a respective one of the original sub-blocks of one of the original blocks with which the projected block received by the pixel unit 5 is associated.

In one implementation, the image generator 11 outputs an enable signal upon receipt of the external image to enable the controller 21 to control the switching module 54 of each of the pixel units 5. The controller 21 receives the enable signal from the image generator 11, and outputs a control signal to the switching module 54 of each of the pixel units 5, so that the switching module 54 is controlled, based on the control signal, to output the total electrical stimulus signal (iₒ) at the output terminals 542 in sequence at periodic intervals.

In another implementation, the image generator 11 outputs an enable signal each time a projected image is outputted thereby, the enable signal being associated with the outputted projected image. For example, the enable signal is one of (M) number of enable signals, which are respectively associated with the (M) number of projected images, and the image generator 11 outputs a selected one of the enable signals depending upon which of the (M) number of projected images is facing outputted. The controller 21 receives the enable signal from the image generator 11, and outputs a control signal upon receipt of the enable signal and according to the enable signal. Specifically, the control signal is one of (M) number of control signals, which are respectively associated with the (M) number of enable signals, and the controller 21 outputs a selected one of the control signals depending upon which of the (M) number of enable signals is received. The switching module 54 of each of the pixel units 5 is controlled to output the total electrical stimulus signal (iₒ) at one of the output terminals 542 associated with the control signal thus received from the controller 21. For instance, if the enable signal with which the control signal is associated is associated with a first one of the projected images, the switching module 54 outputs the total electrical stimulus signal Ltd at a first one of the output terminals 542.

The switching module 54 includes a number (M) of switches 543, each of which is coupled between the input terminal 541 of the switching module 54 and a respective one of the output terminals 542 of the switching module 54, and each of which is operable between an ON state and an OFF state. Only one of the switches 543 is operated in the ON state at one time based on the control signal received from the controller 21.

Upon, outputting one of the projected images associated with the same external image, the image generator 11 outputs the corresponding enable signal to the controller 21, which in turn outputs the corresponding control signal to the pixel array 5, such that the corresponding one of the switches 543 of the switching module 54 of each of the pixel units 51 of the pixel array 5 is operated in the ON state to allow for the total electrical stimulus signal (iₒ) to be outputted at the corresponding output terminal 542 to stimulate the corresponding retinal sub-region 547.

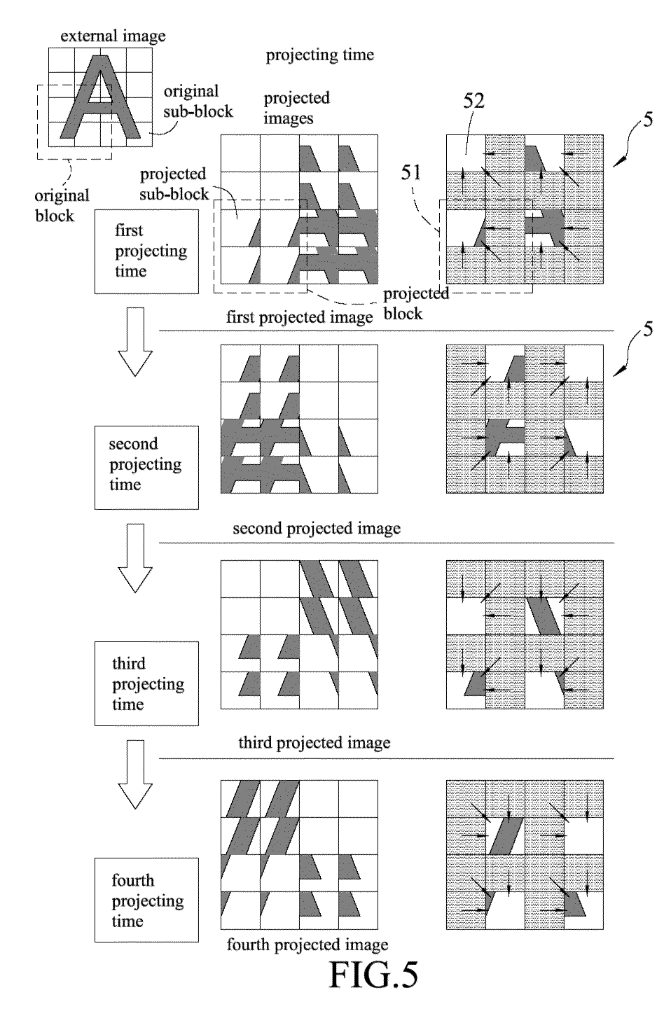

Referring to FIGS. 4 and 5, N=4 and M=4 in the illustrated example. In this example, the external image has four original blocks, each of which has four original sub-blocks, the image generator 11 (see FIGS. 1 and 2) generates and outputs four projected images associated with the external image, each of the projected images has four projected blocks, and each of the projected blocks has four identical sub-projected blocks. The four projected images, namely a first projected image, a second projected image, a third projected image and a fourth projected image, are outputted at a first projecting time, a second projecting time, a third projecting time and a fourth projecting time, respectively. The pixel array 5 includes four pixel units 51, each of which includes four optical sensing modules 52, and in turn, four image detectors 521, and the switching module 54 of each pixel unit 51 includes four switches 543.

At the first projecting time, the first projected image is outputted. For the first projected image, the four identical projected sub-blocks of an upper left one of the projected blocks are associated with an upper left one of the original sub-blocks of an upper left one of the original blocks of the external image, the four identical projected sub-blocks of an upper right one of the projected blocks are associated with an upper left one of the original sub-blocks of an upper right one of the original blocks of the external image, the four identical projected sub-blocks of a lower left one of the projected blocks are associated with an upper left one of the original sub-blocks of a lower left one of the original blocks of the external image, and the four identical projected sub-blocks of a lower right one of the projected blocks are associated with an upper left one of the original sub-blocks of a lower right one of the original blocks of the external image. An upper left one of the pixel units 51 receives the upper left projected block of the first projected image, and outputs the total electrical stimulus signal (iₒ) to stimulate an upper left one of the retinal sub-regions 547 of an upper left one of the regions of the retina by operating the switch 543 connected thereto in the ON state and the rest of the switches 543 in the OFF state based on the control signal from the controller 21. An upper right one of the pixel units 51 receives the upper right projected block of the first projected image, and outputs the total electrical stimulus signal (iₒ) to stimulate an upper left one of the retinal sub-regions 547 of an upper right one of the regions of the retina by operating the switch 543 connected thereto in the ON state and the rest of the switches 543 in the OFF state based on the control signal from the controller 21. A lower left one of the pixel units 51 receives the lower left projected block of the first projected image, and outputs the total electrical stimulus signal (iₒ) to stimulate an upper left one of the retinal sub-regions 547 of a lower left one of the regions of the retina by operating the switch 543 connected thereto in the ON state and the rest of the switches 543 in the OFF state based on the control signal from the controller 21. The lower right one of the pixel units 51 receives the lower right projected block of the first projected image, and outputs the total electrical stimulus signal (iₒ) to stimulate an upper left one of the retinal sub-regions 547 of a lower right one of the regions of the retina by operating the switch 543 connected thereto in the ON state and the rest of the switches 543 in the OFF state based on the control signal from the controller 21.

At the second projecting time, the second projected image is outputted. For the second projected image, the four identical projected sub-blocks of an upper left one of the projected blocks are associated with an upper right one of the original sub-blocks of the upper left original block of the external image, the four identical projected sub-blocks of an upper right one of the projected blocks are associated with an upper right one of the original sub-blocks of the upper right original block of the external image, the four identical projected sub-blocks of a lower left one of the projected blocks are associated with an upper right one of the original sub-blocks of the lower left original block of the external image, and the four identical projected sub-blocks of a lower right one of the projected blocks are associated with an upper right one of the original sub-blocks of the lower right original block of the external image. The upper left pixel unit 51 receives the upper left projected block of the second projected image, and outputs the total electrical stimulus signal (iₒ) to stimulate an upper right one of the retinal sub-regions 54 of the upper left region of the retina. The upper right pixel unit 51 receives the upper right projected block of the second projected image, and outputs the total electrical stimulus signal (iₒ) to stimulate an upper right one of the retinal sub-regions 547 of the upper right region of the retina. The lower left pixel unit 51 receives the lower left projected block of the second projected image, and outputs the total electrical stimulus signal (iₒ) to stimulate an upper right one of the retinal sub-regions 547 of the lower left region of the retina. The lower right pixel unit 51 receives the lower right projected block of the second projected image, and outputs the total electrical stimulus signal (iₒ) to stimulate an upper right one of the retinal sub-regions 547 of the lower right region of the retina.

At the third projecting time, the third projected image is outputted. For the third projected image, the four identical projected sub-blocks of an upper left one of the projected blocks are associated with a lower left one of the original sub-blocks of the upper left original block of the external image, the four identical projected sub-blocks of an upper right one of the projected blocks are associated with a lower left one of the original sub-blocks of the upper right original block of the external image, the four identical projected sub-blocks of a lower left one of the projected blocks are associated with a lower left one of the original sub-blocks of the lower left original block of the external image, and the four identical projected sub-blocks of a lower right one of the projected blocks are associated with a lower left original sub-block of the lower right original block of the external image. The upper left pixel unit 51 receives the upper left projected block of the third projected image, and outputs the total electrical stimulus signal (iₒ) to stimulate a lower left one of the retinal sub-regions 547 of the upper left region of the retina. The upper right pixel unit 51 receives the upper right projected block of the third projected image, and outputs the total electrical stimulus signal (iₒ) to stimulate a lower left one of the retinal sub-regions 547 of the upper right region of the retina. The lower left pixel unit 51 receives the lower left projected block of the third projected image, and outputs the total electrical stimulus signal (iₒ) to stimulate a lower left one of the retinal sub-regions 547 of the lower left region of the retina. The lower right pixel unit 51 receives the lower right projected block of the third projected image, and outputs the total electrical stimulus signal (iₒ) to stimulate a lower left one of the retinal sub-regions 547 of the lower right region of the retina.

At the fourth projecting time, the fourth projected image is outputted. For the fourth projected image, the four identical projected sub-blocks of an upper left one of the projected blocks are associated with a lower right one of the original sub-blocks of the upper left original block of the external image, the four identical projected sub-blocks of the upper right projected block are associated with a lower right one of the original sub-blocks of the upper right original block of the external image, the four identical projected sub-blocks of the lower left projected block, are associated with a lower right one of the original sub-blocks of the lower left original block of the external image, and the four identical projected sub-blocks of the lower right projected block are associated with a lower right one of the original sub-blocks of the lower right original block of the external image. The upper left pixel unit 51 receives the upper left projected block of the fourth projected image, and outputs the total electrical stimulus signal (iₒ) to stimulate a lower right one of the retinal sub-regions 547 of the upper left region of the retina. The upper right pixel unit 51 receives the upper right, projected block of the fourth projected image, and outputs the total electrical stimulus signal (iₒ) to stimulate a lower right one of the retinal sub-regions 541 of the upper right region of the retina. The lower left pixel unit 51 receives the lower left projected block of the fourth projected image, and outputs the total electrical stimulus signal (iₒ) to stimulate a lower right one of the retinal sub-regions 547 of the lower left region of the retina. The lower right pixel unit 51 receives the lower right projected block of the fourth projected image, and outputs the total electrical stimulus signal (iₒ) to stimulate a lower right one of the retinal sub-regions 547 of the lower right region of the retina.

After all four projecting times have passed, the retina is stimulated to acquire a complete vision of the external image.

In view of the above, since the image generator 11 outputs multiple projected triages associated with a single external image in the manner described above with each projected image representing distinct portions of the external image, and since the pixel array 5 combines the electrical stimulus signals (iₛ) associated with the same portion of the external image to obtain the total electrical stimulus signal (iₒ) that is (M) times each electrical stimulus signal (iₛ), the stimulus provided to each retinal sub-region 547 can be relatively large even if the retinal implant device 2 has insufficient power supply.

While the present invention has been described in connection with what is considered the most practical and preferred embodiment, it is understood that this invention is not limited to the disclosed embodiment but is intended to cover various arrangements included within the spirit and scope of the broadest interpretation and equivalent arrangements.

What is claimed is:

1. An artificial retinal system comprising:
an optical device including
an image generator receiving an external image, generating a number (M) of projected images based differently on the external image, and outputting the projected images in sequence through optical projection, the external image having a number (N) of original blocks, each of which has a number (M) of original sub-blocks, each of the projected images having a number (N) of projected blocks, each of which has a number (M) of identical projected sub-blocks, the projected sub-blocks of each of the projected blocks of each of the projected images being associated with a corresponding one of the original sub-blocks of a corresponding one of the original blocks, where N and M are positive integers greater than 1; and
a retinal implant device including
a pixel array including a number (N) of pixel units respectively receiving the projected blocks of one of the projected images, each of said pixel units generating, based on the respective one of the projected blocks thus received, a number (M) of electrical stimulus signals that are respectively associated with the projected sub-blocks of the respective one of the projected blocks, and combines the electrical stimulus signals to obtain a total electrical stimulus signal.

2. The artificial retinal system of claim 1, wherein the projected sub-blocks of an i^{th} one of the projected blocks of a j^{th} one of the projected images generated by said image generator are associated with a one of the original sub-blocks of an i^{th} one of the original blocks of the external image, where i=(1, 2, . . . , N) and j=(1, 2, . . . , M).

3. The artificial retinal system of claim 1, wherein each of said pixel units includes:
a number (M) of optical sensing modules respectively receiving the projected sub-blocks of the respective one of the projected blocks received by said pixel unit, each of said optical sensing modules generating, based on the respective one of the projected sub-blocks thus received, a respective one of the electrical stimulus signals; and
a switching module having an input terminal that is coupled to said optical sensing modules for receiving the electrical stimulus signals respectively therefrom, and a number (M) of output terminals, the electrical stimulus signals being combined at said input terminal to obtain the total electrical stimulus signal, said switching module selectively outputting the total electrical stimulus signal at one of said output terminals that corresponds to a respective one of the original sub-blocks of one of the original blocks with which the projected block received by said pixel unit is associated.

4. The artificial retinal system of claim 3, wherein, said optical sensing modules of each of said pixel units respectively correspond in position to retinal sub-regions of a corresponding region of a retina of an eyeball to which said retinal implant device is installed.

5. The artificial retinal system of claim 3, wherein each of said optical sensing modules includes:
an image detector receiving the respective one of the projected sub-blocks, and converting the respective one of the projected sub-blocks thus received into a current signal; and
a current amplifier coupled to said image detector for receiving the current signal therefrom, and amplifying the current signal to obtain the respective one of the electrical stimulus signals.

6. The artificial retinal system of claim 3, wherein said switching module of each of said pixel units includes:
a number (M) of switches, each of which is coupled between said input terminal of said switching module and a respective one of said output terminals of said switching module, and each of which is operable between an ON state and an OFF state, only one of said switches of said switching module of each of said pixel units being operated in the ON state at one time.

7. The artificial retinal system of claim 3, wherein, said retinal implant device further includes:
a controller coupled to said switching module of each of said pixel units of said pixel array, and controlling said switching module of each of said pixel units to output the total electrical stimulus signal at said one of said output terminals.

8. The artificial retinal system of claim 7, wherein said image generator enables said controller to control said switching module of each of said pixel units to output the total electrical stimulus signal at said one of said output terminals.

9. The artificial retinal system of claim 8, wherein said optical device further includes:
a synchronizer detecting an occurrence of a predetermined event, and generating a reset signal when the occurrence of the predetermined event is detected;
said image generator being coupled to said synchronizer for receiving the reset signal therefrom, said image generator re-receiving the external image upon receipt of the reset signal.

10. The artificial retinal system of claim 7, wherein said retinal implant device further includes:
an optical to electrical converter coupled to said controller and said pixel array, receiving the projected images, and converting the projected images into electricity for powering said controller and said pixel array.

11. The artificial retinal system or claim 10, wherein said optical device further includes:
a light generator emitting an auxiliary light;
said optical to electrical converter further receiving the auxiliary light, and converting the projected images and the auxiliary light into the electricity.

12. The artificial retinal system or claim 11, wherein said output terminals of said switching modules of said pixel units of said pixel array are respectively to be disposed in contact with a plurality of retinal sub-regions of a retina of an eyeball to which said retinal implant device is installed.
